(19) United States
(12) Patent Application Publication
(10) Pub. No.: US 2018/0279948 A1
(43) Pub. Date: Oct. 4, 2018

(54) METHOD AND SYSTEM FOR PUPILLOMETRIC ASSESSMENT OF TOXIN EXPOSURE

(71) Applicant: BAE Systems Information and Electronic System Systems Integration Inc., Nashua, NH (US)

(72) Inventors: John Medberry, Austin, TX (US); Mark J. Dube, McDade, TX (US); John D. Johnson, Thrall, TX (US); Joseph S. McGaughey, Austin, TX (US); Mitchell J. Nord, Round Rock, TX (US); Will R. Grigsby, Austin, TX (US); Gaylan L. Brannan, Bastrop, TX (US); Somit S. Mathur, Austin, TX (US)

(21) Appl. No.: 15/765,913

(22) PCT Filed: Oct. 7, 2016
(86) PCT No.: PCT/US16/55999

§ 371 Date: Apr. 4, 2018

Related U.S. Application Data
(60) Provisional application No. 62/238,287, filed on Oct. 7, 2015.

(51) Int. Cl. A61B 5/00; A61B 3/11; A61B 3/14

(52) U.S. Cl. CPC A61B 5/4845; A61B 3/145; A61B 3/112

(57) ABSTRACT

The system and method of pupil size independent pupillometry used for assessing critical diagnostic characteristics relating to neurotransmission and neuroactivity as applied to toxin exposure. The system and method utilizes the timing of a response to stimuli for a pupil of an individual, characterizes the timed responses, determines the latency of the timed responses, measures the minimum constriction value for the timed responses; and assesses the timed responses collected to diagnose changes in neurotransmission and neuroactivity for an individual exposed to organophosphate (OP) nerve agents or botulinum toxin (BTX).

### CROSS REFERENCE TO RELATED APPLICATIONS

This Application claims the benefit of U.S. Provisional Patent Application No. 62/238,287, filed Oct. 7, 2015, the content of which is incorporated by reference herein in its entirety.

### FIELD OF THE DISCLOSURE

The present disclosure relates to pupillometry and more particularly to a pupil size agnostic method of pupillometry used for assessing critical diagnostic characteristics relating to neurotransmission and neuroactivity for the assessment of toxin exposure.

### BACKGROUND OF THE DISCLOSURE

Existing measurements for pupillometry and the diagnostic characterizations related to it measure the linear or areal aspects of the pupil. Existing methods look at changes over time and apply threshold limits on those values. However, these products report multiple false detects due to the nature of the type of measurement taken. Current methods are also inherently tied to the size of the pupil in the evaluation and do not account for wide variations in pupil size within the population. A variation in pupil size greatly degrades the quality of the measure. Normalization does not exist in these conventional methods that also typically rely on baseline measurements to determine if there has been a change, which may not be available.

### SUMMARY OF THE DISCLOSURE

It has been recognized that current methods of pupillometry are prone to false detects and are inherently biased by the size of the pupil. The techniques of pupillometry of the present disclosure decouple pupil measurements from the size of the pupil and use the timing of the responses, characteristics of the time response, latency, the minimum constriction value, and the like to explicitly provide for a more accurate assessment with fewer false detects.

One aspect of the present disclosure is a pupil size agnostic method of pupillometry used for assessing critical diagnostic characteristics relating to neurotransmission and neuroactivity comprising, timing the response to a stimuli for a pupil of an individual, characterizing the timed response, determining the latency of the timed response, measuring the minimum constriction value for the timed response; and assessing the timed response collected to diagnose changes in neurotransmission and neuroactivity for an individual.

One aspect of the present disclosure is a pupil size independent method of pupillometry used for assessing exposure toxins in an individual comprising, capturing one or more images of a pupil of an individual that is dark adapted; determining a pre-stimulus maximum diameter for the pupil of an individual; providing a stimulus to the pupil of an individual; capturing one or more images of the pupil of an individual following the stimulus; timing the pupil's response to the stimulus; measuring the extent of the pupil's response to the stimulus; determining the onset of constriction for the pupil; measuring the minimum constriction value for the pupil; characterizing the pupil's response to the stimulus; and analyzing the pupil's responses to the stimulus to assess exposure to organophosphate (OP) nerve agents or botulinum toxin (BTX) in an individual, wherein the pupil's responses are pupil size independent.

One embodiment of the method further comprises providing illumination to a pupil of an individual to allow for dark adaptation. Another embodiment of the method is wherein the illumination is provided by one or more IR LEDs for at least 8 s.

An embodiment of the method is wherein the stimulus is provided by one or more visible LEDs for about 70 ms. In some cases, the steps of capturing one or more images of the pupil of an individual is with one or more CCD or CMOS cameras.

Another embodiment of the method is wherein determining the onset of constriction comprises determining the time at which the pupil is 95% dilated. In some cases, the method further comprises assessing the relationship between the pre-stimulus maximum diameter and the minimum constriction value for the pupil to assess toxin exposure in an individual. In other cases, the data is normalized.

In yet another embodiment of the method, detecting exposure to OP nerve agents in an individual is done by determining a Pupil Constriction Rate (PCR) value of 2 or less.

Another aspect of the present disclosure is a system for assessing toxin exposure in an individual comprising, a housing having a subject side and an operator side comprising; one or more eyepieces optically connected to one or more imaging devices; a lighting module configured to control one or more light sources; an imaging module configured to control one or more imaging devices; a memory for storing and retrieving one or more images of one or more pupils of an individual as captured by the imaging module; a system manager module configured to control the light module and the imaging module; and a processing module for assessing exposure to organophosphate (OP) nerve agents or botulinum toxin (BTX) in an individual.

One embodiment of the system further comprises a display coupled to the housing. In some embodiments, the one or more light sources comprises illuminating and stimulating LEDS and the illuminating LEDs are IR LEDs and the stimulating LEDs are visible LEDs.

In another embodiment of the system, the system manager module further comprises rules for controlling the one or more light sources or the one or more imaging devices. In some cases, the processing module is co-located on the system and assesses exposure to organophosphate (OP) nerve agents or botulinum toxin (BTX) in an individual by analyzing one or more images of a pupil of an individual, and in others it is eternal to the system.

In yet another embodiment of the system, a display is external to the housing.

These aspects of the disclosure are not meant to be exclusive and other features, aspects, and advantages of the present disclosure will be readily apparent to those of ordinary skill in the art when read in conjunction with the following description, appended claims and accompanying drawings.

### BRIEF DESCRIPTION OF THE DRAWINGS

The foregoing and other objects, features, and advantages of the disclosure will be apparent from the following description of particular embodiments of the disclosure, as illustrated in the accompanying drawings in which like reference characters refer to the same parts throughout the different views. The drawings are not necessarily to scale, emphasis instead being placed upon illustrating the principles of the disclosure.

FIG. 1 shows a comparison of sample to assessment time for several BTX exposure detection methods.

FIG. 2A shows an anatomical diagram of the neural retina and optic nerve as extensions of the central nervous system (CNS).

FIG. 2B shows a diagrammatic model of the neural retina and optic nerve as extensions of the central nervous system (CNS).

FIG. 3 shows an operator utilizing the device for detection of toxin exposure in an individual according to one embodiment of the present disclosure.

FIG. 4 shows a semi-transparent perspective view of one embodiment of the device of the present disclosure.

FIG. 5 shows one embodiment of the system of the present disclosure.

FIG. 6A shows the variation between the left and the right eye for the same individual for one embodiment of the present disclosure.

FIG. 6B shows normalized data, smoothed and scaled to pre-flash value for one embodiment of the present disclosure.

FIG. 7A shows the correlation of latency and pupil diameter for one embodiment of the present disclosure.

FIG. 7B shows a distribution plot of constriction latency for the left eye and for the right eye according to one embodiment of the present disclosure.

FIG. 7C shows a distribution plot of pupil diameter for the left eye and for the right eye according to one embodiment of the present disclosure.

FIG. 8A shows a correlation between the minimum pupil diameter and the time of the minimum value according to one embodiment of the present disclosure.

FIG. 8B shows a distribution plot of the time of the minimum value for the left eye and for the right eye according to one embodiment of the present disclosure.

FIG. 8C shows a distribution plot of minimum pupil diameter for the left eye and for the right eye according to one embodiment of the present disclosure.

FIG. 9 shows a correlation of the minimum and the maximum values of the extent for one embodiment of the present disclosure.

FIG. 10 shows raw diameter data (mm) plotted over time (seconds) for one embodiment of the present disclosure.

FIG. 11 shows raw diameter data (mm) scaled to pre-flash mean for one embodiment of the present disclosure.

FIG. 12 shows raw diameter data (mm) scaled to pre-flash mean and the minimum is scaled to zero for one embodiment of the present disclosure. It also shows high consistency in the time duration from pre-flash values to minimum values for diameter data according to the principles of one embodiment of the present disclosure.

FIG. 13 shows a histogram of pupil constriction rate (PCR) measurements in areal units (pixel/msec) for one embodiment of the present disclosure.

FIG. 14A and FIG. 14B shows histograms of pupil dilation rate (PDR) measurements in areal units (pixel/msec) at different times after the minimum value has been reached for one embodiment of the present disclosure.

FIG. 15 shows a screenshot of one embodiment of the system according to the principles of the present disclosure.

FIG. 16 shows a flow chart of one embodiment of the system of the present disclosure.

FIG. 17 shows one embodiment of the system of the present disclosure.

FIG. 18 shows a flow chart of the processing for one embodiment of the system of the present disclosure.

### DETAILED DESCRIPTION OF THE DISCLOSURE

Biological nerve agents have existed since the 1930s with the first production and testing taking place during WWII. The threat was not deployed until 1980s beginning with the Iraqi Military attack on Northern Iraqi Kurds followed by the an attack from Iraqi insurgents during the first Gulf War and then an event in 1995 where Sarin gas was released in the Tokyo subway. A recent case of a deployed threat took place during the Syrian Civil War where Sarin gas was released in 2013. These declared and undeclared chemical weapon stockpiles signify potential threats to both our armed forces and our nation via acts of bioterrorism.

One existing analytical system for the detection of biological warfare agents and infectious diseases identifies these agents through the use of clinical specimens in microbiology laboratories. These laboratories, often located in forward deployed combat support hospitals and ships are equipped with common laboratory support equipment such as Class II Bio Safety Cabinet, refrigerator, freezer, level work surfaces, line power sources, lighting, and appropriately trained personnel. The system consists of a liquid sample analytical instrument with a computer and software, consumable assays and reagents, and sample preparation protocols and equipment.

Another assessment regimen for human exposure to organophosphate (OP) nerve agents uses blood cholinesterase (ChE) testing to evaluate the presence of ChE inhibitors. Such testing is often undertaken with a commercially available test kit. While this kit offers a relatively affordable Point of Care solution its methodology remains invasive. It also requires consumable supplies, including reagents that for adequate sensitivity and precision must remain stable in the often varying local temperature and humidity environment. ChE assessments provide a semi-quantitative estimation of ChE activity that is of limited utility in medical surveillance.

Botulinum toxin (BTX) is the most acutely lethal toxin known, with an estimated human median lethal dose (LD₅₀) of 1.3-2.1 ng/kg intravenously or intramuscularly and 10-13 ng/kg when inhaled. BTX can cause botulism, a serious and life-threatening illness in humans and animals. It is a serious bioterrorism threat because of its lethality and ease of manufacture. As a bioterrorism threat, BTX need not be delivered in contaminated food or water but can be delivered in aerosol form. In this case the onset of symptoms may take longer than symptoms arising from food-borne poisoning.

The gold standard for identification of BTX is the in vitro Enzyme-Linked Immunosorbent Assays (ELISAs) followed by confirmation diagnosis from a mouse lethality assay.

A summary of the issues related to current BTX tests are provided below in Table 1.

| Test Name | Methodology | Drawbacks |
|---|---|---|
| Mouse | Intraperitoneal | Sample to Result Cycle Time of 4 |
| Lethality | injection | Days. |
| Assay | of mice with diluted | Labor intensive—cannot be automated |
| | suspected | Requires laboratory with animal |
| | BTX sample | facility |
| | and observation of | |
| | symptoms. | |
| ELISA | In Vitro assay | Sample to Result Cycle Time of 5 to 6 |
| | based on | hours |
| | binding antibody | Cannot discriminate between |
| | to the | functionally active and inactive forms |
| | toxin. Sample source | of BTX |
| | patient blood | Capture antibody must be specific to |
| | or feces. | toxin |
| | | Some foods interfere with ELISAs and |
| | | degrade sensitivity and must be |
| | | determined on an individual basis |
| | | Requires confirmation by Mouse |
| | | Lethality Assay. |

Pupillometry has been used in subject assessment for a host of potential negative states; these include, but are not limited to, exposure to neuro-active toxins, traumatic brain injury, and basic health states such as hypertension, multiple sclerosis, and the like. Generally, a pupilometer measures the mean pre-stimulus value of the pupil and then the pupil is measured over time for the duration of the assessment. A review of previously collected data showed considerable variation in pupil size within the human population. It was also clear that with the range of resultant constriction, a viable, consistent measure was not determinable using current methods. For example, certain state of the art products reported false detections a great deal of the time (e.g., reported that a person had been exposed to neurotoxin when in fact they had not been). The present techniques are divorced from the extent measurement of the pupil. In certain examples, this is done using feature extraction from the pupil extent versus time to provide a consistent measure with lower variability. In one embodiment of the present disclosure, feature extraction on the onset of response (e.g., constriction) and at the minimum extent (i.e., at its most constricted) provides a consistent measure for this particular set of physiological responses. By extracting features in this way, dependency on the extent of the pupil is removed and gives a more reliable metric for assessment.

Existing measurements for pupillometry and the diagnostic characterizations related to it measure the linear or areal aspects of the pupil as a function of time and apply threshold limits on those values. Current methods typically rely on a pupil measurement and a baseline pass/fail criteria based upon a threshold of the time rate of change of the pupil extent. This inherently carries the size of the pupil in the evaluation. The variation in pupil size greatly degrades the quality of the measure.

In contrast, methods of pupillometry of the present disclosure decouple measurements of the size of the pupil and use the timing of the responses, characteristics of the time response, latency, the minimum constriction value, and the like to explicitly provide a more accurate assessment. In certain embodiments, the statistical improvement (tightening of the distribution) of the method of the present disclosure compared to other implemented methods is about an order of magnitude improvement.

One aspect of the present disclosure is a population independent parameter for pupil response evaluation. The present method is of high intrinsic value due to its consistency and it provides an assessment of neurological related response changes that allow for very high confidence assessment with very low false failures.

It is understood that existing methods do not provide the robust level of detection or the same level of consistency as described herein. Existing methods establish the measurement of the pupillary response based on a measurement of pupil motility (time rate of change of the diameter, area or radius), measurement of the constricted and dilated pupil, and other similar methods. All of the values inherently rely on the pupil of the individual, and these responses are compared to a prior established rate. However, the inclusion of the extent measurement requires inclusion of the subject-dependent measure of the pupil. Even self-rectified values (e.g., using percent) include an inherent linear extent. Further, basing the measure on the extent of the pupil constrains two otherwise independent biometric measures into one which further makes for a biased measurement.

The basic characteristics involved in the extent measurement are the unstimulated maximal pupil extent and the amount of available constriction. The maximal pupil extent has a wide range of variation within the population. The amount of available constriction is coupled to both the population variation and the degradation of motility over the lifetime of an individual. A measurement that is consistent removes both of these variations from the evaluation and still provides useful diagnostic information.

The method presented herein relies on the basic underlying mechanisms in the pupillary response. The governing response to a stimulus of light is dictated by one set of muscles to constrict the pupil. The governing response in the absence of this stimulus is a counter set of muscles to widen the opening of the pupil. In certain embodiments, the independent characteristics of these two sets of responses are used for population independent measurements along with the precise timing of the action of these responses.

It is understood that in the absence of background visible light, the pupil opens to an asymptotic wide clear aperture. As this change in pupil size is governed by the weaker dilation muscle group, measurement of this asymptote is generally convolved with other effects within the individual. For example, measurement of a time spread mean within the point over time (e.g., measure of the extent averaged over time) provides a self-referenced comparative, base value. Application of a stimulus, e.g., a visible light source, for a brief period, activates the constriction (miosis) driven by the constriction muscle group. Measurement of the extent over time (at each time sample) is required. In one embodiment of the present disclosure, the time series of the measurement of the extent is used as a point from which to extract independent values. The onset of constriction, or the constriction latency, is one of such independent values.

In one embodiment of the present disclosure, regardless of the clear aperture base value, the time for latency was consistent within the unaffected population evaluated. In one embodiment, a second parameter that is independent of the population is the time of the minimum compression. These are time dependent values which are independent of the sample. These values are specifically related to neurotransmission and neuroaction values, regardless of pupil size.

Some of the benefits of the system of the present disclosure are found in Table 2.

| Features | Benefits |
|---|---|
| Application of variable | Non-invasive screening for toxins |
| intensity infrared | completed in 4 minutes |
| and visible light to the eye and | |
| measurement of pupillary | |
| response to assess exposure. | |
| Automated test sequence | User intervention not required; may |
| | be effectively operated by non-clinical |
| | personnel |
| Compact, lightweight | Minimal impact on load out of |
| ergonomic design | medic/corpsman |
| | Minimal logistic footprint |
| | Adapted to use on the “dirty” side of |
| | the contamination line |
| Adaptable to binocular | Binocular hardware/software |
| light application/ | configuration increases capabilities to |
| image capture configuration | include assessment of Traumatic |
| | Brain Injury (TBI) |
| Autonomous operation | Consumables such as reagents not |
| | required |
| | Support equipment such as |
| | refrigeration not required |

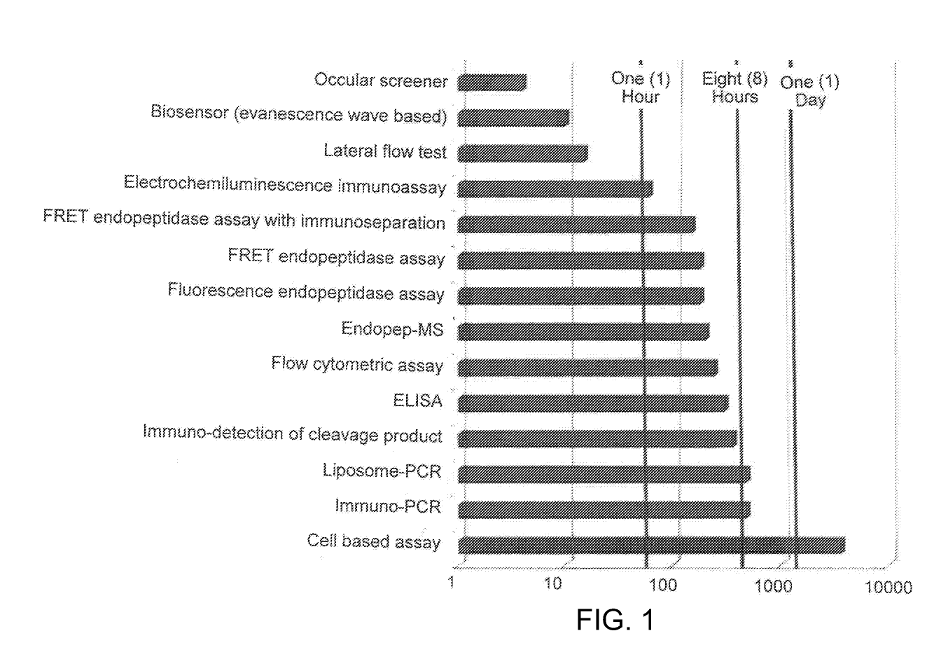

Referring to FIG. 1, a comparison of sample to assessment time for several BTX exposure detection methods is shown. More particularly, many of the current methods take between 1 hour and 8 hours to produce a result and some take over a day. Additionally, the majority of methods require a skilled technician to run the diagnostic test. In contrast, the ocular screening system of the present disclosure does not require particular training to use and the results are much more rapid than any other test providing for better outcomes and treatment protocols as the toxin exposure can be identified quickly. This system also does not require a traditional laboratory or consumables.

The system of the present disclosure provides a non-invasive solution for pre-symptomatic and post symptomatic assessment of poisoning by OP Nerve Agents and BTX. A prolific amount of current scientific literature identifies the eye as one of the most prominent indicators for various hazards and/or diseases to which individuals are exposed. The sensitivity of the eye's reaction to a wide variety of chemicals/toxins and its role as a gauge for internal homeostasis (e.g., cardiovascular and neurophysiological imbalances) has been extensively researched via many scientific disciplines. New techniques and equipment are both harnessing and utilizing this information to define a modern approach to the field of non-invasive early detection of a vast range of physical abnormalities, injuries, and illnesses. Early detection provides an invaluable tool in the subsequent success of treating such conditions.

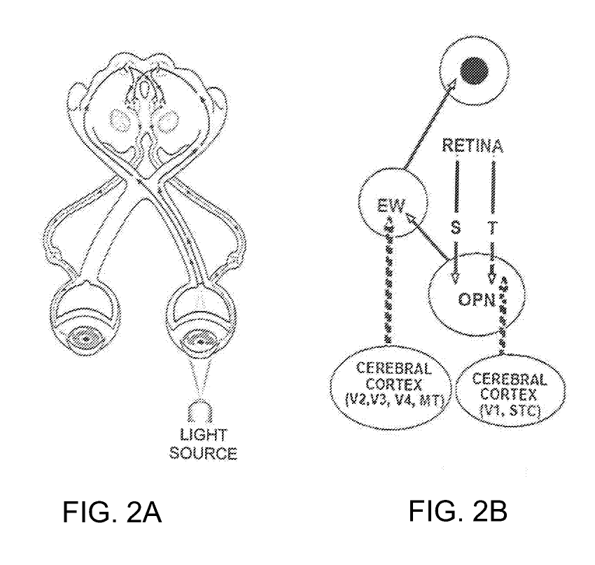

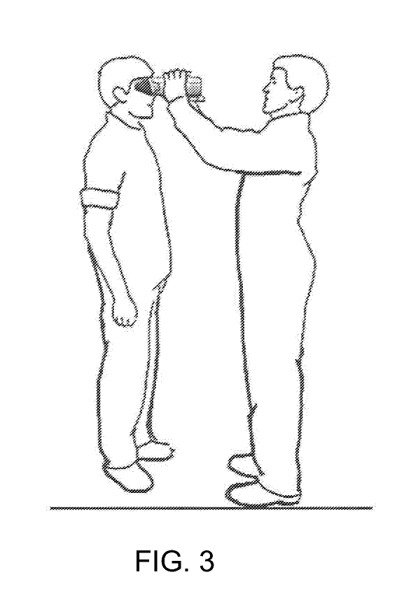

The health and function of the eye reflects the general health of the body as a whole. For example, diseases that compromise the cardiovascular system are detectable via the ocular vasculature, which is clearly visible through the front of the eye. As indicated in FIG. 2A, the neural retina and optic nerve are extensions of the central nervous system (CNS) and therefore serve as an outwardly visible means of monitoring and testing the function of this system. The lymphatic system circulates through the eye via its connections to the non-vasculature fluids of the eye, often depositing traces of untoward chemicals or metals. In addition, the cornea is an extremely sensitive ectodermal tissue that serves as an indicator for agents that also affect the skin. Thus, numerous medical references indicate and support that the many systemic effects of a variety of toxic materials/chemicals or the effects of diseases and genetic defects may be detected by careful and informed examination of the eye.

Poisoning by nerve agents disrupts the mechanism by which nerves transfer messages to organs and leads to miosis (the contraction of pupils), an early biomarker of exposure. BTX poisoning prevents the release of acetylcholine presynaptically and thus blocks neurotransmission. This results in flaccid paralysis of autonomic nerves, always beginning with the cranial nerves. Some symptoms include double and blurred vision due to mydriasis (the dilation of the pupil).

Referring to FIG. 2B, a diagrammatic model of the neural retina and optic nerve as extensions of the central nervous system (CNS) is shown. More particularly, the paths of modulation from the several areas of the cerebral cortex are shown. There, V1 is the Primary Visual Cortex (or visual area 1), V2 is visual area 2, and so on. The Middle Temporal (MT) is also known as V5. The Primary Visual Cortex is responsible for processing all visual stimuli. STC represents the Superior Temporal Cortex and the Edinger-Westphal (EW) nucleus moderates pupil dilation and aids (via parasympathetic fibers) in convergence of the eyes and lens adjustment. Information about an image from an eye is transmitted to the brain along the optic nerve (OPN). Different populations of ganglion cells in the retina send information to the brain through the optic nerve.

The system of the present disclosure is intended to operate in a field screening site providing limited environmental shelter. In this field environment subjects may have been exposed to a nerve agent in the last 24 to 48 hours. Transportation of the device to and from the field site may include the use of light wheeled vehicles, small watercraft, fixed-wing aircraft, or rotary-wing aircraft. The device may also be transported by pack animal or on foot by the operator for more than 5 miles as the unit is lightweight and weather and impact resistant.

Subjects coming to the field screening site may not be able to walk. Therefore, screening of subjects who may be sitting or lying down is expected. Also, subjects may lack the strength or muscle function due to exposure symptom onset to hold the device in their own hands raising the risk that the subject might drop the device during a screening activity. Thus, the device may be held and controlled by an operator during screening as shown in FIG. 3. The subject will need to remove glasses, goggles, or head coverings that interfere with the face-to-device interface but the operator does not need to be clinically trained to utilize the device.

When a subject is first brought to the operator, the subject will provide the operator with a name or other identifying information and the operator will observe and record the approximate age, gender, and other observable physical metrics of the subject. The operator will also briefly question the subject about their exposure experience and possible location of the exposure. This data collection process will take approximately 2 minutes. The ocular screening system will then be used to screen the subject for symptoms of nerve agent or BTX exposure. The system will collect ophthalmological biomarker data from the subject for approximately 30 seconds and then analyze the data for an additional 90 seconds before providing the operator with a value that can be used to make a determination of exposure severity. With an average screening time of 4 minutes, a screening rate of 12 persons per hour is expected.

Using the method of the present disclosure requires some way to maintain the position of the subject's eyes relative to the device's image detector or compensate for variations in position. This may be accomplished several ways, either through a mechanism (e.g., a human head to device interface), optical design, or via an algorithm (e.g., measuring and correlating any change in size to the subject's facial features which do not change as rapidly as the pupil, such as the iris diameter or the distance from the inner and outer corner of the eye).Other methods may include a sonar sensor, sterographic imaging and/or triangulation, a 3D image sensor with depth of field, variable focus and/or zoom optics, or the like.

The ruggedized device of the present disclosure is designed to be a portable, handheld instrument. In some embodiments, the device is powered by common, commercially available Nickel Metal Hydride AA cells. In some cases, an external battery charger is supplied in a transit/storage container. The device is approximately 6″×6″×3″ in size and weighs approximately 3 lbs. The device's housing is environmentally sealed.

In certain embodiments of the system of the present disclosure, the operator interface is simple; only an On/Off pushbutton switch and a Start Screening pushbutton switch are required. Acquired data may be exported with the device serving as a USB Device via the USB 2.0 protocol to a USB Host. In some embodiments, the data may be transmitted wirelessly. Real time screening results may be presented to the operator on a Liquid Crystal Display (LCD), or other display.

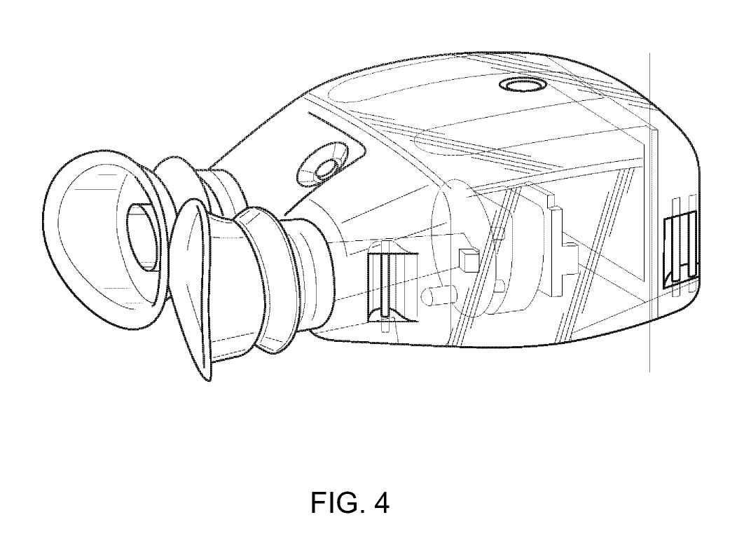

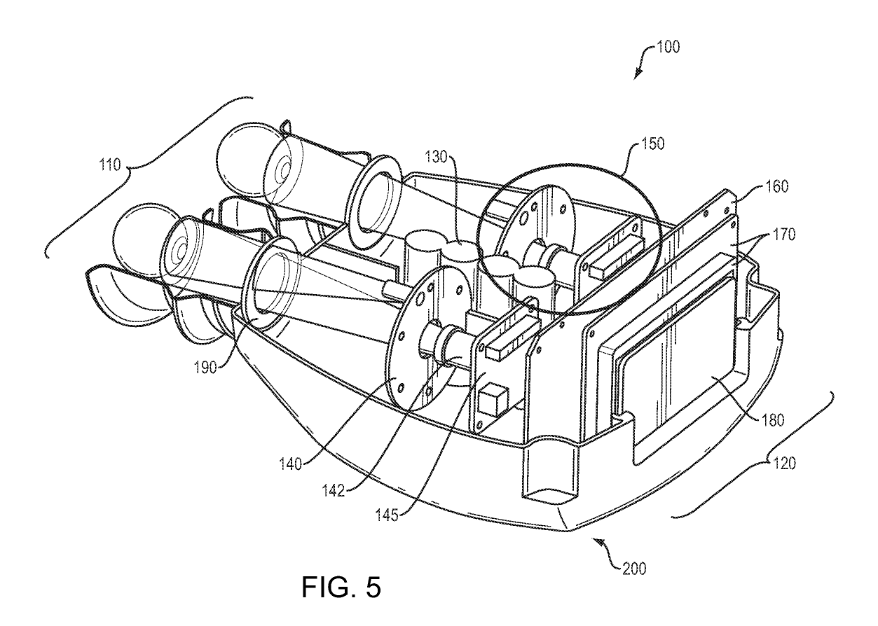

Referring to FIG. 4, an external semi-transparent view of one embodiment of the system of the present disclosure is shown. Referring to FIG. 5, one embodiment of the device with a housing removed is shown. More particularly, the system is comprised of a binocular device 100 having a subject side 110 and an operator side 120. In certain embodiments, the device is configured to view the response of pupils in one or both eyes upon stimulation of one or the other eye. In one example, the device is configured to stimulate (e.g., flash visible light) the left eye. The device may be contained in weather proof housing 200 in order to weather the external environment. In some cases, the device is ruggedized to withstand impact and harsh conditions in the field. In this example, the system has an onboard power supply, such as batteries 130 but can also be powered by external connection to a power source.

The device has light sources 140 to stimulate one or both eyes. In certain embodiments, the light sources can be IR LEDs or visible LEDs. The device may be equipped with image detectors for one or both eyes 145. In certain embodiments, the device has optical windows or screening to protect the detector lenses from physical damage 190.

The device in this example has optics 142 in front of the imager to improve zoom and focus. The system can be coupled to a processing unit that employs a computer program to process the data. The device in a further example can transmit the data from the detector via wireless technology to a processing unit that is no co-located with the system. There is a Circuit Card Assembly (CCA) in this example that contains digital electronics 160 for capturing, storing, and processing the data within the system. A modular imaging unit or camera assembly 150 is used to capture the images of the pupil during operation. On the operator side, this embodiment has a display 180 such as a liquid crystal display (LDC) with associated printed circuit board 170 to show the results of the processing to a user.

The system of the present disclosure provides a precise, sensitive and repeatable test that does not require the need for a trained clinician. Algorithms have been developed to monitor pupillary light response for the detection of OP nerve agents and BTX. The algorithms declare BTX detected when the algorithm reports pupil latency (from the point of contraction back to the surface area corresponding to 90% of the largest surface area) greater than or equal to 400 ms. The algorithms provide an assessment of nerve agent exposure as a function of pixel/msec change in constriction, also known as Pupillary Constriction Rate (PCR). Those threshold values are shown, below, in Table 3:

| | | PCR | PDR |
|---|---|---|---|
| Toxin | Exposure Level | (pixels/msec) | (pixels/msec) |
| Organophosphate | No Exposure | 4-6 | 0.3-1.0 |
| | Potentially Exposed | <4-2 | <0.3 |
| | Severe Exposure | <2 | <0.1 |

In one embodiment of the system, the following is a procedure used for detecting BTX and OP nerve agents.
I. Pre-Test Phase
   a. Device powered on
   b. Testing options selected
   c. Device placed over eyes of subject
   d. Testing triggered
II. Testing Phase/Loop
   a. Infrared (IR) lights turned on; Continuous image acquisition of eye initiated; Every image analyzed for pupil size; (x) duration delay to allow for dark adaptation of pupils and for automated focusing by device
      i. Determination of baseline static pupil area (SPA; mm) determined for each eye based on final acceptable (by the algorithm) image prior to step b, below
   b. Visible wavelength light of intensity(i) applied to pupil for a duration (t)
   c. (y) duration delay
      i. Based upon images acquired and analyzed for pupil area during light application (step b) AND during the delay period (step c), the following are determined
         1. Pupil reaction latency (PRL; msec)=time between light initiation and pupil constriction/dilation, determined by a xx % change in pupil size from SPA
         2. Magnitude of maximal pupil area change (MPA; mm²)=difference between SPA and area at point of maximal change (RA) {dilation/largest area; constriction/smallest area}
         3. Percent of maximal pupil area change (PPA)=MPA divided by SPA
         4. Pupil reaction duration (PRD; msec)=time between beginning of pupil reaction (after xx % change from baseline) and time of maximal change (dilation/largest area; constriction/smallest area)
         5. Rate of pupil reaction (PRR; mm²/msec)=MPA divided by PRD
         6. Pupillary Constriction Rate (PCR; pixels/msec). Change in pupillary pixels as a function of time from dark adaption to minimum extent.
         7. If multiple reactions occur (pupillary constriction and dilation) prior to initiation of the next step (d), the first to occur will have the above 5 values determined (PRL, MPA, PPA, PRD and PRR) and the extent of the second will be determined by measuring the area of the pupil at the point of maximal change (RA) and MPA, PPA, PRD and PRR with respect to RA (instead of SPA; i.e., substitute RA for SPA in above equations)
   d. Depending on the options selected, the next step will be:
      i. Test ended

In another embodiment of the system, the following is a procedure used for detecting BTX and OP nerve agents.

On the first frame of the flash, the Find ROI Edges function runs to find a region of interest smaller than 160×160 that contains the pupil. The function goes thru every pixel in the (30, 30) to (130,130) region (specific values are determined by the KEEP_OUT_ROI definitions). It records min and max pixels, then divides the spread in the pixel values by 5 and adds the minimum pixel value to that to obtain an initial threshold below which is considered the pupil value for ROI search. Then it scans through the same region by row and by col to determine the location of the row and col respectively with the maximum number of values below that threshold. That (row, col) coordinate is used as an initial pupil edge search point.

Starting from that edge search point, it does a search in all four directions using a 7 point edge kernel. The kernel looks for 3 non-pupil pixels above the threshold, and 4 pupil pixels below the threshold to determine an edge. It does this edge find from the starting coordinate and the outside of the image frame, and checks to see if both edges match. If they don't match, the next row or col respectively is chosen and the search is repeated. If they do match that point is considered one of the four corner edges.

Once four edges have been found, it adds a 10 pixel constant and uses that as the new reduced ROI. If that ROI is smaller than 50×50, or has an edge that is on one of the 160×160 image frame corners, it will find a new ROI again on the next frame. Otherwise it will use that ROI consistently throughout the whole pupil analysis segment.

The next function is a search within the ROI to create a 256 bin histogram of the pixel values with 4 pixel values per bin starting at the lowest pixel value count. It finds a maximum peak in the first 87 bins, and then searches the histogram after that peak for three pixel bins that fall below ⅓ of that peak pixel count. The (pixel value+50) represented by that edge bin is considered the threshold value for determined whether a pixel is pupil or not. Next, a search is done from both the left hand side and right hand side of all the rows in the ROI region to find the first pixel value below that threshold and its coordinates at that point are added as a potential circle point.

Those circle points are then filtered by first taking the average x and y value as the circle mid-point then rejecting any point that lies outside the average value of the radius squared by 0.64 and 1.44 (80% and 120% respectively of the average radius). This filtering is done twice and then the average radius of the filtered points is used to calculate the surface area of the pupil circle (PI*Average Radius Squared). The diameter (or radius) is then tracked over time after each measurement. A least-squares fit between maximum and minimum extents is applied to calculate PCR. A threshold is applied to PCR to assess exposed, potentially exposed, and not exposed.

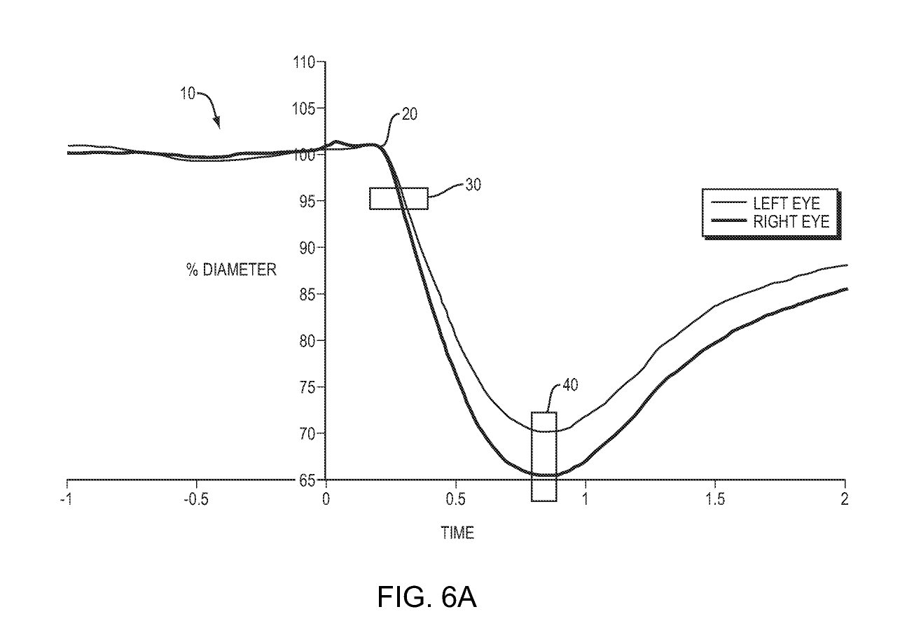

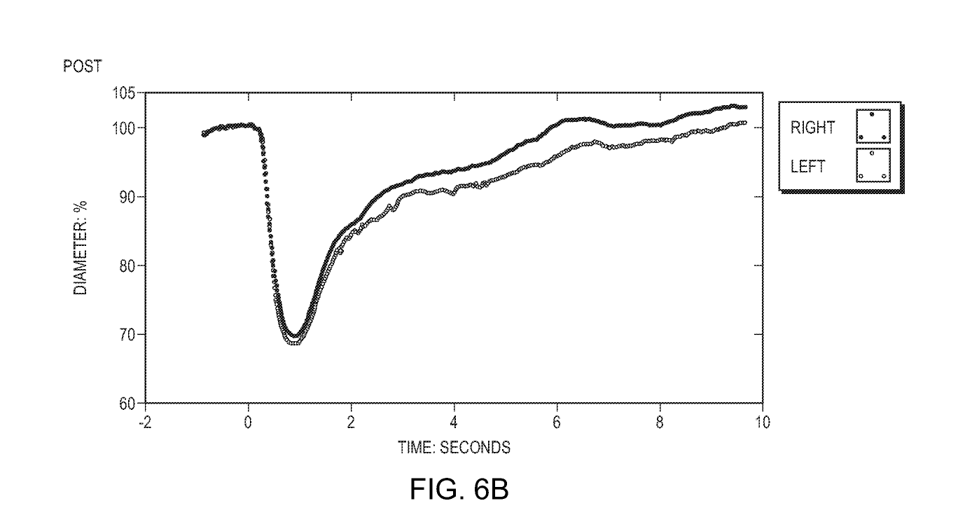

Referring to FIG. 6A, plots of a left and a right eye responding to a stimulus are shown. The x-axis represents time (in seconds) with time 0 representing the visible flash. The flash occurs after 8 seconds of dark adaptation (IR LED exposure). In certain embodiments, the IR LEDs are on continuously. In certain embodiments, the IR LEDs have a radiant intensity of about 20 mW/sr. In certain embodiments, the IR LEDS are always on. The y-axis represents the % of pupil diameter, with 100% representing the pupil diameter prior to an applied stimulus 10. In certain embodiments, the 100% value represents the diameter of the pupil after dark adaption for about 8 seconds. In certain embodiments, the applied stimulus is a 70 ms flash of visible light from visible LEDs. In certain embodiments, the dark adaption duration and flash duration can be varied. In certain embodiments, the visible LEDs have a luminous intensity (Iv) of about 2063 cd. In certain embodiments, the wavelength of the visible LEDs is about 850 nm. In certain embodiments, 20 represents the inflection point following the flash. In certain embodiments, the onset of constriction 30 is taken at 95% diameter. In certain embodiments, variation between a left eye's and a right eye's response to a stimulus 40 is present to some degree. Ideally, there wouldn't be any variation, because the consensual response is perfect. But under normal conditions, people don't have identical responses.

Referring to FIG. 6B, normalized data, smoothed and scaled to pre-flash value is shown. More specifically, both left and right eyes were sampled, and the plots illustrate a consensual response because both pupillary responses track each other with minimum variation. This was normalized to the pre-flash value and measurement was in linear extent units (%). In this example, both the left and the right eyes track in a consensual manner, which is indicative of normal population. In one example, the duration of test is 18s, with a total of (18 s*80 samples/sec)=1440 samples (or images) per eye.

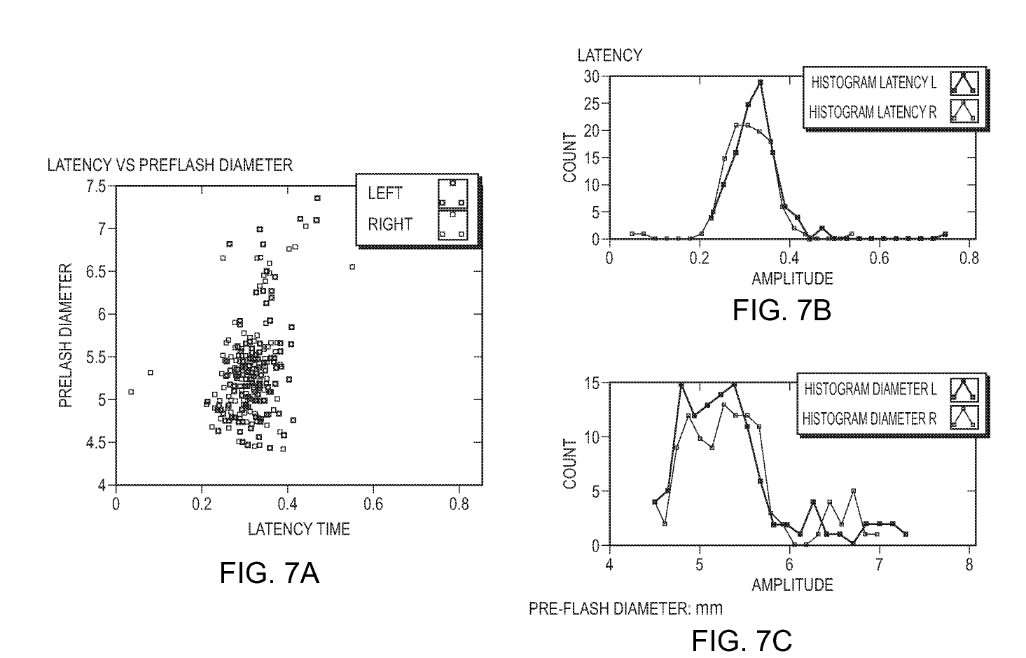

Referring to FIGS. 7A-7C, evaluation of the constriction latency, or onset of constriction, and evaluation of the correlation to the pupil diameter are shown for each eye. Review of the data, from a statistical viewpoint, shows significant independence of the latency time from the diameter. Referring to FIG. 7A, no specific correlation between the latency time and the pre-flash pupil diameter is found. Referring to FIG. 7B and FIG. 7C, the amplitude of the latency response shows a very Gaussian type (normal) distribution, and the spread of the pupil diameter within the population shows great variation, respectively.

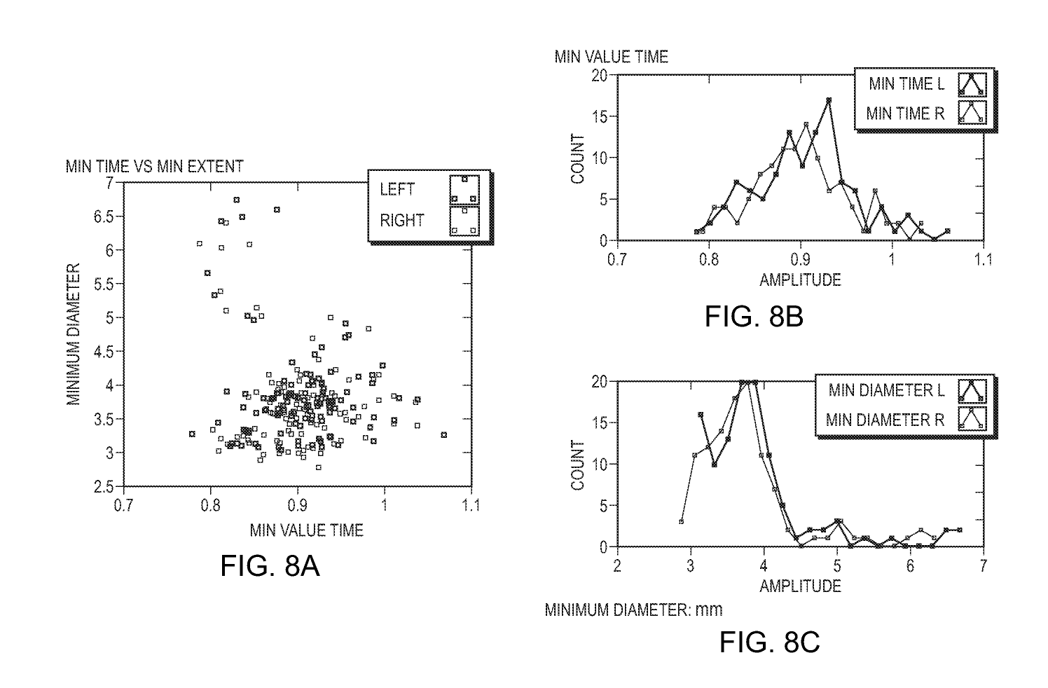

Referring to FIGS. 8A-8C, evaluation of the minimum extent and the time at minimum and the related correlations are shown. In FIG. 8A, the minimum value (i.e., smallest diameter) and the time of the minimum value is shown to be de-correlated from each other. On FIGS. 8B and 8C, the latency response histogram shows a very Gaussian type (normal) distribution, and the spread of the diameter is indicative of the population variation.

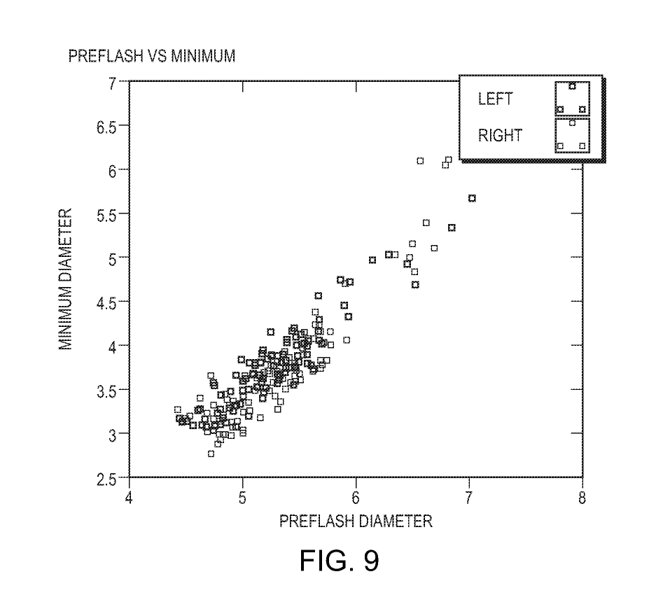

Referring to FIG. 9, correlation of the minimum diameter values and the maximum (pre-flash) diameter values of the pupil extent are shown. More specifically, the measurement of the rate of change is shown to be intrinsically related to the maximum value and the minimum value. FIG. 9 shows the correlation is strongly linear between pre-flash mean (e.g., widest diameter) and the minimum value attained (e.g., following the flash). In certain embodiments, the linear correlation of the maximum and the minimum is exploited, where a deviation from this trend is another characteristic that is independent of the variation in pupil size in a given population.

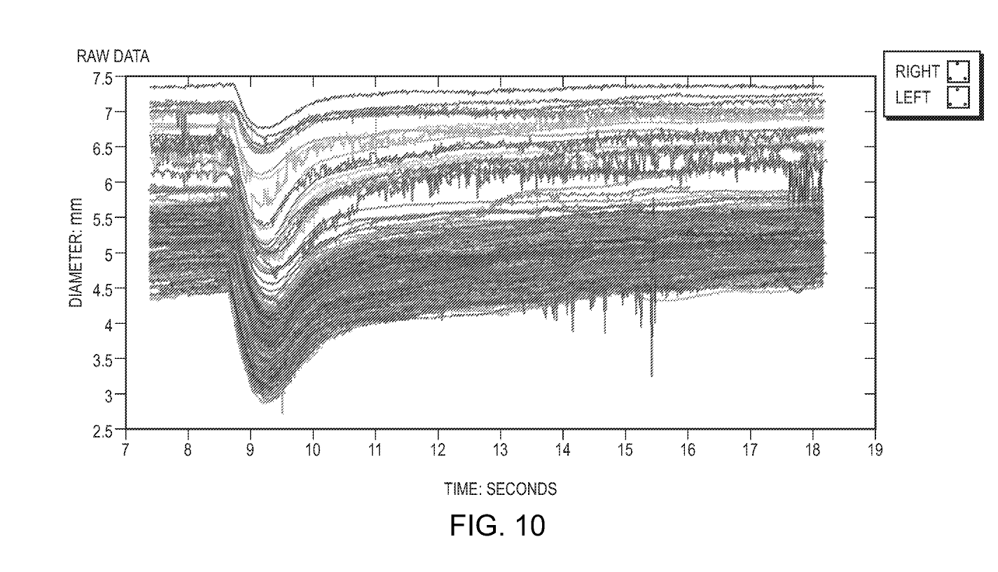

Referring to FIG. 10, raw diameter data is shown. More specifically, the vertical axis diameter is in millimeters and the horizontal is time in seconds. As can be seen, the consistency of the data, regardless of the measured diameter is very prevalent, with the response, in time, characteristic on every trace. In certain embodiments, initial diameter (after dark adaption) varies within the population and the data must be normalized. In certain embodiments, the slope to the minimum extent varies within the population as well.

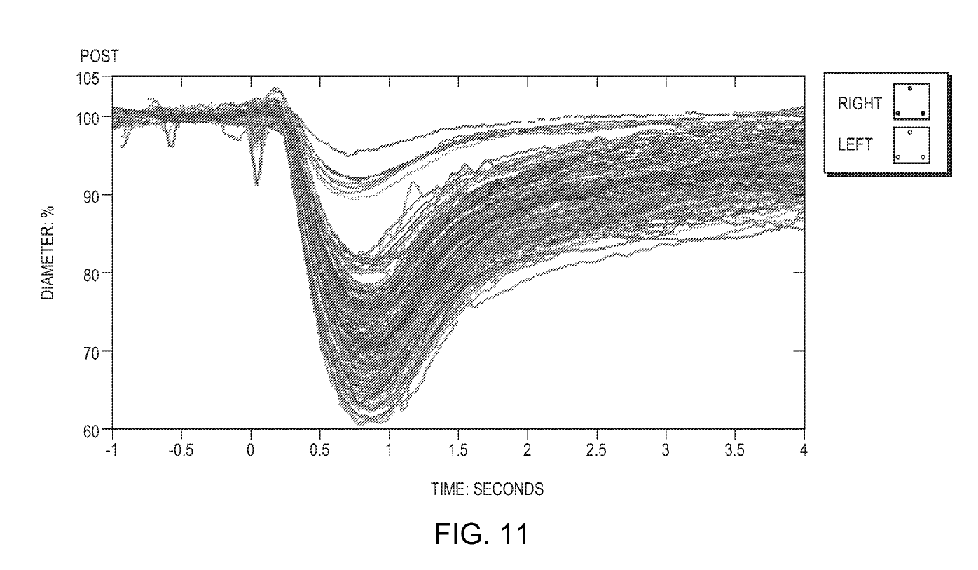

Referring to FIG. 11, raw data resealed with the minimum at zero and pre-flash mean at 1 is shown. The initial step in post processing is normalization to the pre-stimulus extent. Data shown in FIG. 11 used a 0.5 second duration mean divided through all other extent values. The range of slopes to the minimum is distinct.

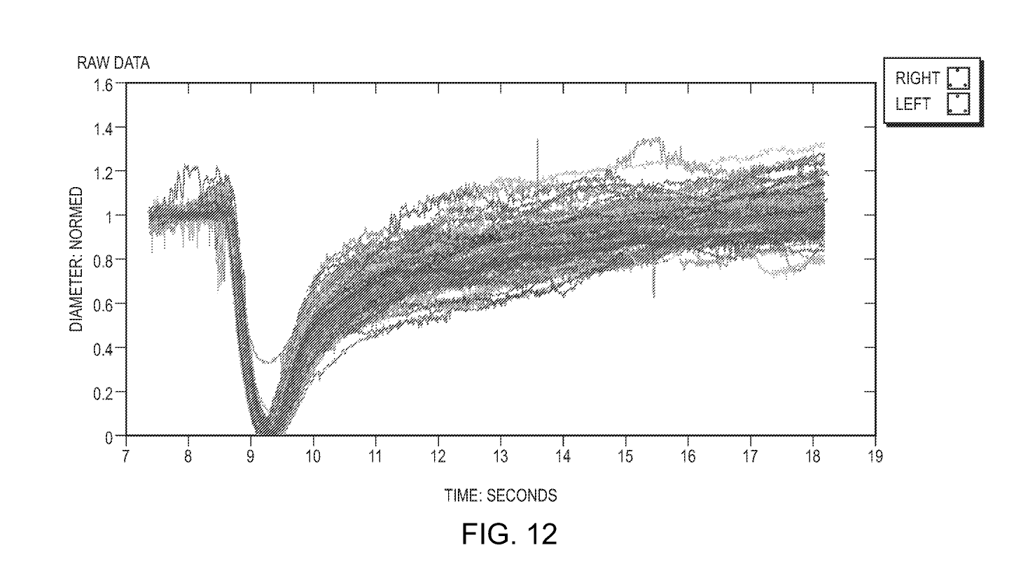

Review of the data presented in FIG. 12 shows high consistency in the time duration from pre-flash values to a minimum value. This is a function of the previously stated independence of the response on base extent.

From evaluation of the data presented so far, one question remained, ‘is there an equally independent evaluation possible for dilation?’ In certain embodiments, it is possible to segment pupil dilation/constriction into two time domain governed responses—an immediate impulse response followed by a minimum pupil diameter and then weak recovery. In certain embodiments, there is diversity in the time domain and amplitude of the recovery and it is inherently more condition dependent. The general trend appears to be a hand off between musculature responses, essentially from the cessation of the application of the constriction muscle group to the start of control by the dilation muscle group.

According to certain testing, multiple individuals were assessed utilizing the following method: for all times an image was acquired of the eye; eyes were dark adapted, illuminated only by IR LEDs (e.g., NIR about 850 nm) for 8 seconds.; and at 8 seconds, a 70 ms visible light flash from a white LED was applied to one eye. In certain embodiments, the system incorporated dual eye measurement and the data included both eyes. Data was collected for 10 additional seconds at a sampling rate of about 80 Hz. In certain embodiments, the pixel resolution for the apparatus was about 0.08 mm/pixel. The pupil extent was measured from the imagery. In one example, the diameter was in millimeters. The response of the diameter of the pupil was plotted as a function of time. In one embodiment, the diameter versus time data was processed to measure the constriction latency and the time to minimum value. In certain embodiments, the point in the data at which the diameter was 95% of the maximum value was measured from the time series as the onset of constriction. The time of the minimum value was also extracted from the time series. In certain embodiments, a fitting function was used for smoothness in the minimum.

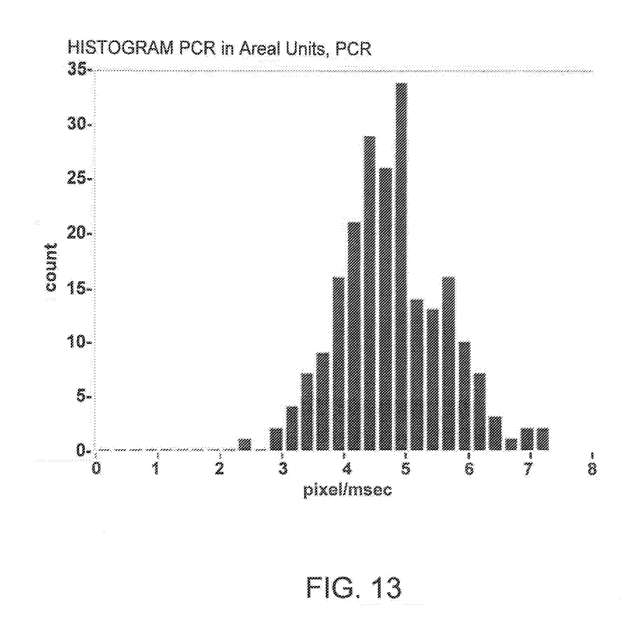

Referring to FIG. 13, raw rescaled data is used to determine the pupil constriction rate (PCR) in pixels/msec, by using the min value response and then stepping back (in time) from that value a region is selected for a linear slope evaluation. FIG. 13 shows a histogram of this evaluation. The wide spread of the results is indicative of the coarseness of the measure. While no explicit dependence on mean pupil size was seen in this evaluation, the diverse scatter of this range shows the broad swath of the figure of merit.

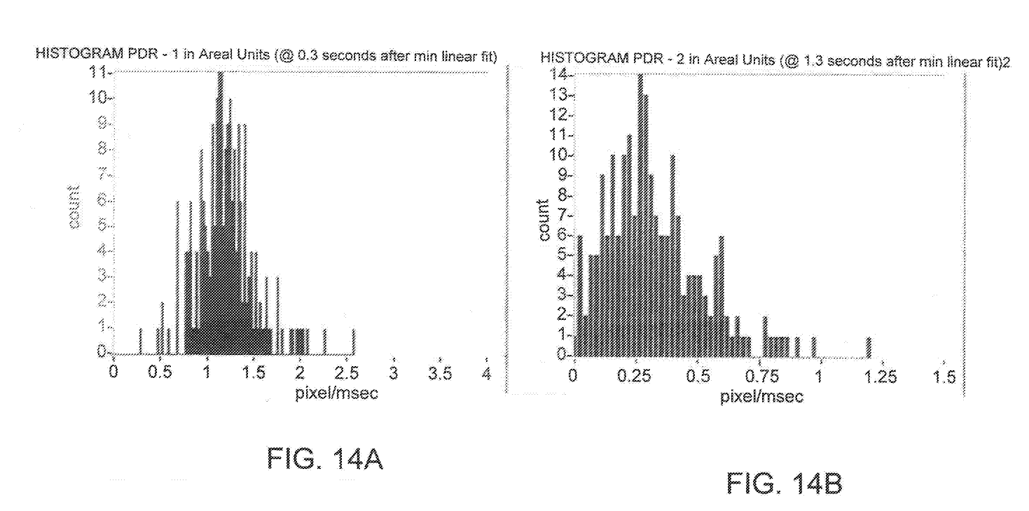

In FIG. 12, above the transition from fast response to slower recovery pupil dilation rate (PDR) is evident in the curve of recovery. However this is a ‘soft transition’ that is not simply extricable. In order to do so, the slope extracted at 0.3 seconds after the minimum value was observed. This corresponded to a PDR-I value for the immediate dilation. In certain embodiments, the OSI device has metrics related to PDR that are on the order of 0.1 pixels/msec or less that indicate definite exposure. Further, the slope of recovery after 1.3 seconds from the minimum value was a second PDR that characterized the second response (PDR-2). FIG. 14A and FIG. 14B shows these two histograms: one for PDR-I and one for PDR-2 and clearly illustrates that a small population sample can have wide variation. On the OSI OSCAR unit, the reported values for PDR were considered poor (at least on version 29 and 27) and this was likely due to the sensitivity of the image processing routine. Improved measurements are possible.

In one embodiment, using Table 3, above, and PCR values, of 218 data points sampled, 16% show up as exposed at some level. Using Table 3, and PDR-I values, 1% of individuals were exposed at some level. Using Table 3, and PDR-2 values, 59% of individuals were exposed. The current identified definition of PDR may not be sufficient to know which value was being reported. It is conceivable that some chemicals could influence only one value or the other in terms of the measured latency and minimum response time.

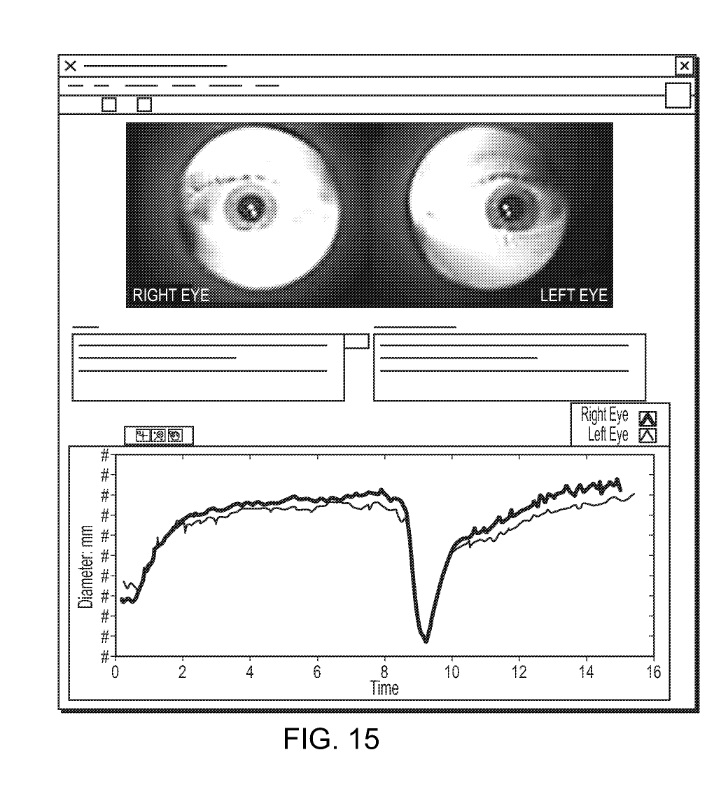

Referring to FIG. 15, a screenshot of one embodiment of the assessment system according to the principles of the present disclosure is shown. More particularly, the system comprises a graphical user interface (GUI) where analysis of the data can be viewed and assessed. In certain embodiments, the GUI is viewable on a portable device, such as a smart phone. In another embodiment, the GUI is resident on a laptop computer that is connected to the system via USB interface. There are several features which make the present approach unique and effective: 1) the system simultaneously captures ocular imagery from both eyes, enabling a quantitative assessment of consensual response; 2) the blockage of ambient light from both eyes isolates the consensual response to the stimulus created by the visible light flash in the system; and 3) the normalization of both measurements creates pupillary signatures that improve quality of measurement and statistical relevance.

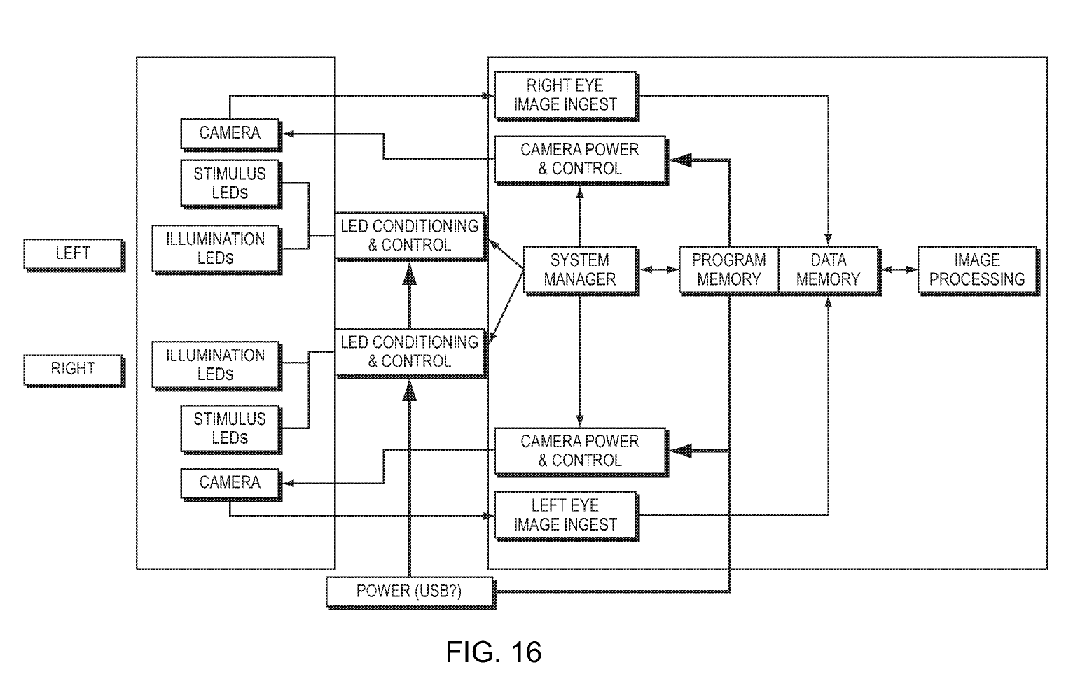

Referring to FIG. 16, a diagrammatic view according to one embodiment of the system is shown. A binocular device is deployed proximate the face of the user to capture information from one or more eyes. In one example the system stimulates one eye while in another embodiment the system stimulates both eyes. There are one or more illumination light sources such as LEDs or lasers for each eye to provide the stimulation. These illumination LEDs can be in the NIR range and provide a simulated darkness while still allowing for image capture of the eye at rest. In certain embodiments, the system has one or more stimulus LEDs for each eye. In a further embodiment the stimulus LEDs are in the visible range and provide a “white” flash.

Still referring to FIG. 16, in certain embodiments the system has a camera for each eye such as CCD or CMOS cameras. In this example the device has LED modules for controlling the illumination LED brightness and duration. For example, the LED modules control the visible LED brightness and duration of flash. The LED control modules can be modifiable by the user. In one embodiment, a set of rules is present such that the device has a set protocol for controlling the LEDs. In other embodiments, there is a system manager for controlling the one or more LEDs and the one or more cameras. In other embodiments, the device has camera modules for controlling the one or more cameras and can be modifiable by the user. The device can also have power modules for controlling the power of the device. In certain embodiments, the power is USB. In certain embodiments, a set of rules is present such that the device has a set protocol for controlling the one or more LEDs and/or the one or more cameras.

Still referring to FIG. 16, in certain embodiments, the system has program memory. In certain embodiments, the system has data memory. In certain embodiments, the device process images on-board. In certain embodiments, the system has an embedded digital processor.

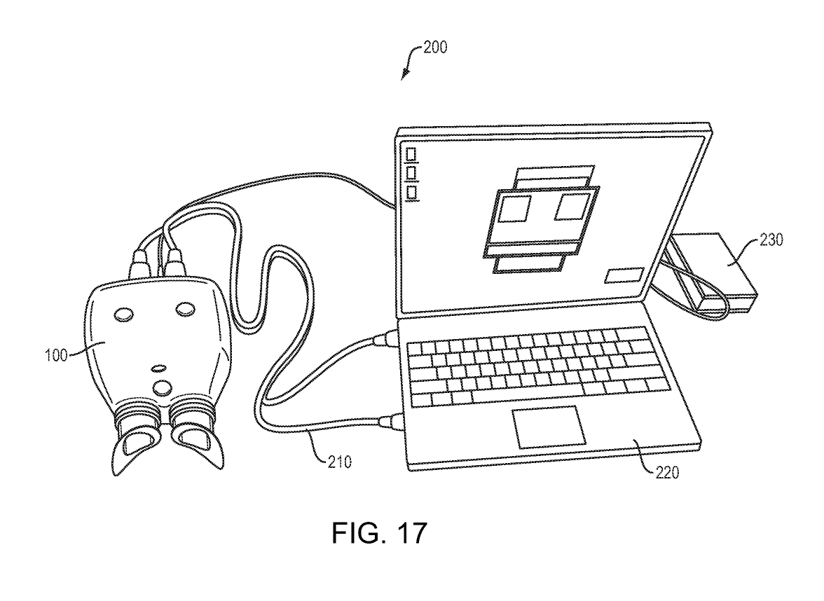

FIG. 17 shows an embodiment of the system of the present disclosure. This embodiment of the system has acquisition hardware 230 in communication with a laptop or other acquisition computing device 220. The system also has a binocular device 100 for use with an individual. In this example, the binocular acquisition device is connected via cables 201 to the acquisition computing device 220. In some systems, the connection is wireless. In other systems, a separate computing system is used for analyzing the data and assessing toxin exposure in an individual. In some cases the analysis is completed remote from acquisition.

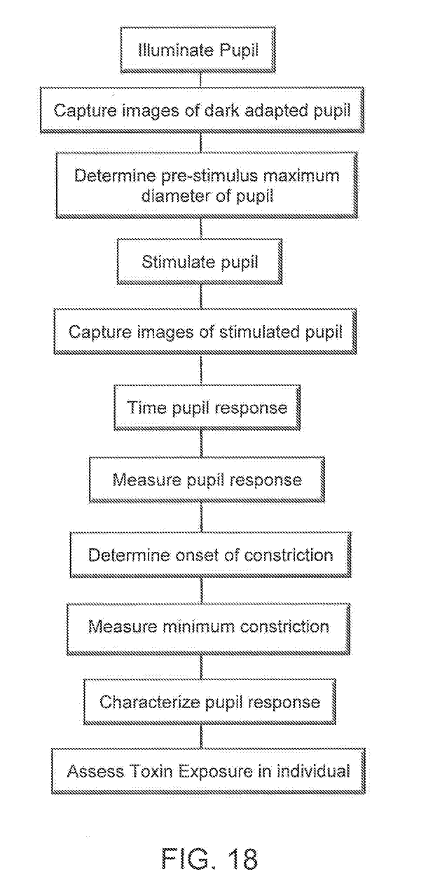

FIG. 18 is a flow chart for the processing according to one embodiment. Here, one or more pupils are illuminated and become dark adapted. Then, images are captured of the one or more pupils. A pre-stimulus maximum diameter for the one or more pupils is determined. A stimulus is applied to the one or more pupils and images are collected of the stimulated pupil over a period of time. Over time, the one or more pupils will return to a dark adapted state if illuminated, as above. The timing of each pupil's response is determined as well as the minimum extent of the constriction for each pupil. The pupil's response is categorized and an assessment is completed to determine if an individual has experienced toxin exposure. At that point, some form of alert is also possible to indicate exposure. In some case, an alert is an audio or visual alert. In some case, an alert is sent as a message.

While the principles of the disclosure have been described herein, it is to be understood by those skilled in the art that this description is made only by way of example and not as a limitation as to the scope of the disclosure. Other embodiments are contemplated within the scope of the present disclosure in addition to the exemplary embodiments shown and described herein. Modifications and substitutions by one of ordinary skill in the art are considered to be within the scope of the present disclosure.

What is claimed:

1. A pupil size independent method of pupillometry used for assessing exposure toxins in an individual comprising,
capturing one or more images of a pupil of an individual that is dark adapted;
determining a pre-stimulus maximum diameter for the pupil of an individual;
providing a stimulus to the pupil of an individual;
capturing one or more images of the pupil of an individual following the stimulus;
timing the pupil's response to the stimulus;
measuring the extent of the pupil's response to the stimulus;
determining the onset of constriction for the pupil;
measuring the minimum constriction value for the pupil;
characterizing the pupil's response to the stimulus; and
analyzing the pupil's responses to the stimulus to assess exposure to organophosphate (OP) nerve agents or botulinum toxin (BTX) in an individual, wherein the pupil's responses are pupil size independent.

2. The method of claim 1, further comprising providing illumination to a pupil of an individual to allow for dark adaptation.

3. The method of claim 2, wherein the illumination is provided by one or more IR LEDs for at least 8 s.

4. The method of claim 1, wherein the stimulus is provided by one or more visible LEDs for about 70 ms.

5. The method of claim 1, wherein the steps of capturing one or more images of the pupil of an individual is with one or more CCD or CMOS cameras.

6. The method of claim 1, wherein determining the onset of constriction comprises determining the time at which the pupil is 95% dilated.

8. The method of claim 1, further comprising assessing the relationship between the pre-stimulus maximum diameter and the minimum constriction value for the pupil to assess toxin exposure in an individual.

9. The method of claim 1, further comprising normalizing the data.

10. The method of claim 1, further comprising detecting exposure to OP nerve agents in an individual by determining a PCR value of 2 or less.

11. A system for assessing toxin exposure in an individual comprising, a housing having a subject side and an operator side comprising; one or more eyepieces optically connected to one or more imaging devices;
a lighting module configured to control one or more light sources;
an imaging module configured to control one or more imaging devices;
a memory for storing and retrieving one or more images of one or more pupils of an individual as captured by the imaging module;
a system manager module configured to control the light module and the imaging module; and
a processing module for assessing exposure to organophosphate (OP) nerve agents or botulinum toxin (BTX) in an individual.

12. The system of claim 11, further comprising a display coupled to the housing.

13. The system of claim 11, wherein the one or more light sources comprises illuminating and stimulating LEDS.

14. The system of claim 13, wherein the illuminating LEDs are IR LEDs and the stimulating LEDs are visible LEDs.

15. The system of claim 11, wherein the system manager module further comprises rules for controlling the one or more light sources or the one or more imaging devices.

16. The system of claim 11, wherein the processing module is co-located on the system and assesses exposure to organophosphate (OP) nerve agents or botulinum toxin (BTX) in an individual by analyzing one or more images of a pupil of an individual.

17. The system of claim 11, wherein the processing module is external to the system and assesses exposure to organophosphate (OP) nerve agents or botulinum toxin (BTX) in an individual by analyzing one or more images of a pupil of an individual.

18. The system of claim 11, further comprising a display external to the housing.
